(19) United States
(12) Patent Application Publication
(10) Pub. No.: US 2019/0015575 A1
(43) Pub. Date: Jan. 17, 2019

(54) PERITONEAL DIALYSIS DEVICE FOR ANIMALS

(71) Applicant: Benepet Co., Ltd., Taipei City (TW)

(72) Inventor: Ta-Lun Tan, Taipei City (TW)

(73) Assignee: Benepet Co., Ltd., Taipei City (TW)

(21) Appl. No.: 15/877,709

(22) Filed: Jan. 23, 2018

(30) Foreign Application Priority Data
Jul. 11, 2017 (TW) 106210194

(51) Int. Cl. A61M 1/28; A61M 39/10; A61M 39/28

(52) U.S. Cl. CPC A61M 1/285; A61M 2250/00; A61M 39/28; A61M 39/105

(57) ABSTRACT

A peritoneal dialysis device includes: a dialysate catheter that is connected at a first end to a dialysate container connector and at a second to a four-way joint; an injection catheter that is connected at a first end to an injection catheter connector and at a second end to the four-way joint; a waste fluid catheter that is connected at a first end to a waste fluid container connector and at a second end to the four-way joint; and a syringe catheter that is connected at a first end to a syringe and at a second end to the four-way joint.

### TECHNICAL FIELD

The invention relates to a peritoneal dialysis device for animals, and more particularly to a peritoneal dialysis device for animals which can accurately, stably and automatically inject the dialysate.

### BACKGROUND

Peritoneal dialysis is a therapy method for clearing metabolic wastes in the blood of patients with renal failure. The method is performed by injecting dialysate into the peritoneal cavity of the patients with renal failure for a period of time and using the peritoneum as a dialysis semipermeable membrane to remove and transport the body wastes and water to the dialysate in the peritoneal cavity, and then draining out the waste fluid containing the body wastes and water, to achieve the purpose of clearing the metabolism wastes in the blood. When practicing the peritoneal dialysis, the patients have to repeat practicing the skill of fluid injection and exchange while learning dialysis-related care, before they can independently perform home peritoneal dialysis therapy.

However, when applying peritoneal dialysis to animals, a variety of difficulties are encountered. Due to different sizes of animals, the amount of dialysate to be injected is not the same. Particularly, small animals are very sensitive to the amount of dialysate injected, which may cause harm or even death to the animal as long as there is an error in the injection amount. In addition, animals are not able to express their own feelings and inform the care worker when they feel discomfort, which further increases the accuracy requirement of the amount of the dialysate injected. The traditional peritoneal dialysis device for human body can not accurately quantify the amount of the dialysate injected, which may easily cause harm to animals. As smaller animal has smaller peritoneal cavity and requires smaller amount of dialysate injected, applying the traditional peritoneal dialysis device for human body on small animal and pushing the dialysate only by gravity, would make the dialysate flow unstable or even stagnant. Due to the animal's inability to perform peritoneal dialysis on its own, the peritoneal dialysis must be performed and supervised by human care workers, thus consuming a great deal of manpower.

### SUMMARY

One or more embodiments disclosed herein provide a peritoneal dialysis device for animals which can accurately quantify the amount of the dialysate injected, slowly but continuously and stably inject the dialysate, and automatically inject the dialysate without the need for human care worker to perform and supervise personally, thus saving a lot of manpower.

In one aspect, one or more embodiments disclosed herein relate to a peritoneal dialysis device that includes:
a dialysate catheter that is connected at a first end to a dialysate container connector and at a second end to a four-way joint;
an injection catheter that is connected at a first end to an injection catheter connector and at a second end to the four-way joint;
a waste fluid catheter that at a first end is connected to a waste fluid container connector and at a second end to the four-way joint; and
a syringe catheter that at a first end is connected to a syringe and at a second end to the four-way joint.

In one aspect, the dialysate catheter is connected to a dialysate container through the dialysate container connector; the injection catheter is connected to a peritoneal catheter through the injection catheter connector; and the waste fluid catheter is connected to a waste fluid container through the waste fluid container connector.

In one aspect, each of the dialysate container connector, the injection catheter connector and the waste fluid container connector is a luer taper.

In one aspect, the injection catheter comprises a waterwheel that rotates in response to the flow of fluid in the injection catheter.

In one aspect, a waste fluid catheter comprises a sampling port.

In one aspect, the dialysate catheter further comprises a dialysate catheter clamp that is disposed on the dialysate catheter and opens or closes a passageway of the dialysate catheter; the injection catheter comprises an injection catheter clamp that is disposed on the injection catheter and opens or closes a passageway of the injection catheter; the waste fluid catheter comprises a waste fluid catheter clamp that is disposed on the waste fluid catheter and opens or closes a passageway of the waste fluid catheter.

In one aspect, the dialysate catheter comprises a multi-way joint including at least three ends, wherein at least two of the at least three ends are connected to a plurality of the dialysate container connectors, and a remaining of the at least three ends is connected to the four-way joint.

In one aspect, the dialysate catheter is connected to a plurality of dialysate containers through the plurality of the dialysate container connectors.

In one aspect, the injection catheter connector is connected to the peritoneal catheter through an isolating short tube to prevent the injection catheter connector from being directly connected to the peritoneal catheter.

In the peritoneal dialysis device for animals of one or more embodiments disclosed herein, the dialysate is drawn up into the syringe before it is injected into the peritoneum, and the amount of the dialysate is precisely quantified on the scale of the syringe, so as to overcome the low accuracy of the conventional peritoneal dialysis devices. Then the care worker or machine slowly but stably injects the dialysate of the syringe into the peritoneal cavity through the catheters and connectors by applying a steady force to the plunger of the syringe to overcome the instability that exists within peritoneal dialysis devices that rely on gravity to push the fluid in the catheter. When the syringe is installed on a machine which can apply a stable force to push the plunger of the syringe slowly and at a constant speed, the peritoneal dialysis device for animals according to one or more embodiments disclosed herein can automatically inject the dialysate without the need for human care worker to perform and supervise personally, thus saving a lot of manpower.

### BRIEF DESCRIPTION OF DRAWINGS

FIG. 1 is a schematic diagram of the peritoneal dialysis device for animals according to one or more embodiments.

FIG. 2 is a flow chart of the method for using the peritoneal dialysis device for animals according to one or more embodiments.

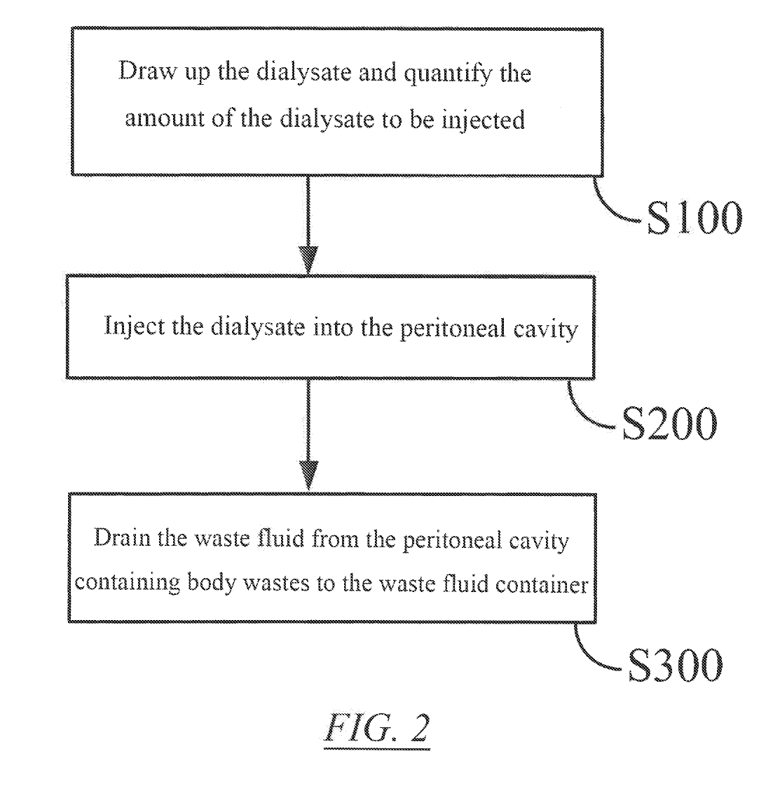

### LIST OF REFERENCE CHARACTERS

1 peritoneal dialysis device for animals
10 dialysate catheter
11 dialysate container connector
12 dialysate catheter clamp
13 multi-way joint
20 injection catheter
21 injection catheter connector
22 injection catheter clamp
23 waterwheel
24 peritoneal catheter
25 isolating short tube
30 waste fluid catheter
31 waste fluid container connector
32 waste fluid catheter clamp
33 waste fluid container
34 sampling port
40 syringe catheter
41 syringe
50 four-way connector

### DETAILED DESCRIPTION

One or more embodiments disclosed herein will be further described in detail with reference to the drawings. The following description is intended to explain the details of one or more embodiments, hut one or more embodiments is not limited to the following description.

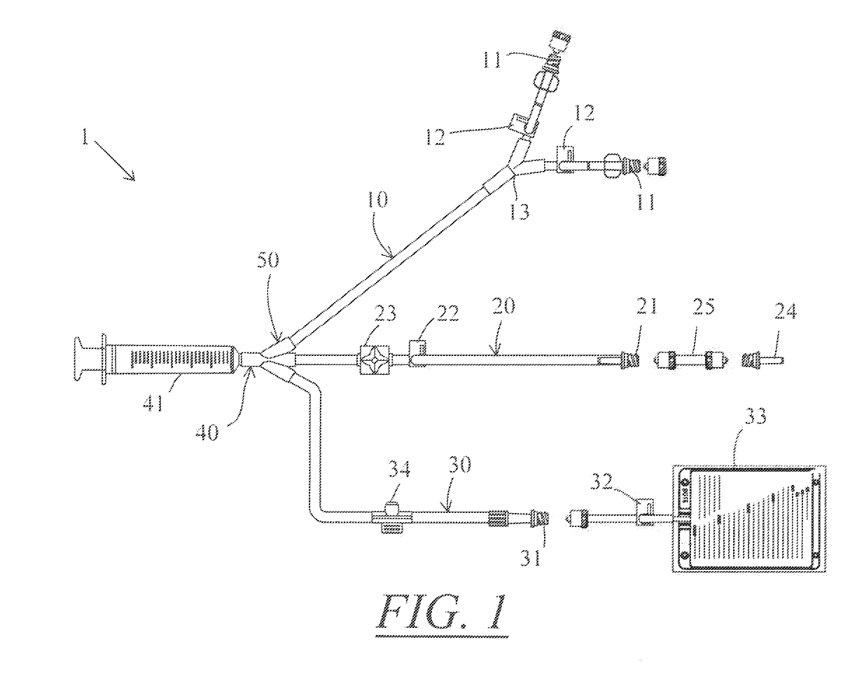

Referring to FIG. 1, FIG. 1 is a schematic diagram of the peritoneal dialysis device for animals according to one or more embodiments. A peritoneal dialysis device for animals 1 according to one or more embodiments includes a dialysate catheter 10, an injection catheter 20, a waste fluid catheter 30, and a syringe catheter 40. One end of the dialysate catheter 10 is connected to a dialysate container connector 11 and the other end of the dialysate catheter 10 is connected to a four-way joint 50. One end of the injection catheter 20 is connected to an injection catheter connector 21 and the other end of the injection catheter 20 is connected to the four-way joint 50. One end of the waste fluid catheter 30 is connected to a waste fluid container connector 31 and the other end of the waste fluid catheter 30 is connected to the four-way joint 50. One end of the syringe catheter 40 is connected to a syringe 41 and the other end of the syringe catheter 40 is connected to the four-way joint 50.

The dialysate catheter 10 is used for transporting the dialysate flowing out from a dialysate container to the direction of the syringe 41. The dialysate catheter 10 is connected to a dialysate container (not shown in the figure) through the dialysate container connector 11, and the dialysate flowing out from the dialysate container sequentially passes through the dialysate container connector 11, the dialysate catheter 10, the four-way joint 50 and the syringe catheter 40 before entering into the syringe 41.

In one or more embodiments, the dialysate container connector 11 is a liter taper. In the peritoneal dialysis device for animals 1 shown in FIG. 1, the portion of the dialysate container connector 11 connected to the dialysate catheter 10 is a male connector; and the portion of the dialysate container connector 11 connected to the dialysate container 10 is a female connector. Alternatively, the portion of the dialysate container connector 11 connected to the dialysate catheter 10 may also be a female connector, and the portion of the dialysate container connector 11 connected to the dialysate container 10 may also be a male connector. In one or more embodiments, the dialysate catheter 10 further comprises a dialysate catheter clamp 12 disposed on the dialysate catheter 10 and opens or closes the passageway of the dialysate catheter 10. However, it may not be necessary to use the catheter clamp 12 to open or close the passageway of the dialysate catheter 10. The catheter may also be manually squeezed by a machine or a care worker to close the passageway of the dialysate catheter 10, or controlled by a valve disposed inside the dialysate catheter 10 to open or close the passageway of the catheter 10 (not shown).

In one or more embodiments, the dialysate catheter 10 comprises a multi-way joint 13 that connects a plurality of dialysate catheter branches. The multi-way joint 13 comprises three or more ends, wherein two or more ends are connected to a plurality of the dialysate container connectors 11, and the other one end is connected to the four-way joint 50, as shown in FIG. 1. The dialysate catheter 10 is connected to a plurality of dialysate containers (not shown in the figure) through the plurality of the dialysate container connectors 11. Thus the peritoneal dialysis device for animals 1 may be connected to a plurality of dialysate containers. As soon as the dialysate in one dialysate container is used up, another dialysate container will back up.

The injection catheter 20 is used for transporting the dialysate flowing out from the syringe 41 to the direction of the peritoneum. The injection catheter 20 is connected to a peritoneal catheter 24 through the injection catheter connector 211. The peritoneal catheter 24 is a catheter of which one end is implanted into the peritoneal cavity of the patient, for introducing external dialysate into the peritoneal cavity of the patient, allowing the dialysate to contact the patient's peritoneum for a period of time. Using the peritoneum as a dialysis semipermeable membrane, the body wastes are removed and transported to the peritoneal dialysate, and thereafter the waste fluid containing the body wastes is drained out through the peritoneal catheter 24. The dialysate from the syringe 41 sequentially passes through the syringe catheter 40, the four-way joint 50, the injection catheter 20, the injection catheter connector 21 and the peritoneal catheter 24 before entering into the peritoneal cavity of the patient.

In one or more embodiments, the injection connector 21 is a luer taper. Since one end of the peritoneal catheter 24 is directly implanted inside the peritoneal cavity of the patient, once the connector at the other end of the peritoneal catheter 24 is contaminated when the peritoneal dialysis device is being installed or replaced, a new operation must be performed to replace the contaminated peritoneal catheter 24 with a new peritoneal catheter 24 for implantation in the patient. This causes unnecessary risk and workload. In order to avoid possible contamination when the connecting of the injection catheter 20 to the peritoneal catheter 24, in one or more embodiments, the injection catheter connector 21 is connected to the peritoneal catheter 24 through an isolating short tube 25, such that the injection catheter connector 21 is prevented from being connected directly to the peritoneal catheter 24. Therefore, when connecting the injection catheter 20, only the isolating short tube 25 will be contacted, thus preventing the injection catheter 20 or the care worker from directly contacting the peritoneal catheter. Even if contamination occurs, one only need to replace the isolating short tube 25 without having to re-surgically replace the peritoneal catheter 24. In the peritoneal dialysis device for animals 1 shown in FIG. 1, the injection catheter connector 21 may be a male connector, and both ends of the isolating short tube 25 may be female connectors, and the connector of the peritoneal catheter 24 contacting the isolating short tube may be a male connector. As the connector of the injection catheter 21 and the connector of the peritoneal catheter 24 contacting the isolating short tube may both be male connectors, therefore the two connectors cannot be connected directly, thus preventing cross-contamination. In one or more embodiments, the injection catheter connector 21 may also be a female connector, and the connectors at both ends of the isolating short tube 25 may also be male connectors, and the connector of the peritoneal catheter 24 contacting the isolating short tube may be a female connector.

In one or more embodiments, the injection catheter 20 further comprises an injection catheter clamp 22 disposed on the injection catheter 20 and opens or closes the passageway of the injection catheter 20. However, it may not he necessary to use the catheter clamp 12 to open or close the passageway of the injection catheter 20. The catheter may also he manually squeezed by a machine or a care worker to close the passageway of the injection catheter 20, or controlled by a valve disposed inside the injection catheter 20 to open or close the passageway of the catheter 20 (not shown).

In one or more embodiments, the injection catheter 20 comprises a waterwheel. 23 that rotates in response to the flow of fluid in the injection catheter 20. For example, when the passageway of injection catheter 20 is open and the fluid therein is flowing, the fluid pushes the waterwheel 23 to rotate. When the passageway of the injection catheter 20 is closed and the fluid therein is stagnant, the waterwheel 23 cannot be pushed and rotated. Therefore, the care worker may monitor the flow of the fluid in the injection catheter 20 by observing the rotation of the waterwheel 23, thus preventing the stagnation of the dialysate flow.

The waste fluid catheter 30 is used for transporting the waste fluid flowing out from the peritoneal cavity to the direction of the waste fluid container 33. The waste fluid catheter 30 is connected to the waste fluid container 33 through the waste fluid container connector 31. The waste fluid flowing out from the peritoneal cavity sequentially passes through the peritoneal catheter 24, the injection catheter connector 21, the injection catheter 20, the four-way joint 50, the waste fluid catheter 30, and the waste fluid container connector 31 before entering into the waste fluid container 33.

In one or more embodiments, the waste fluid container connector 31 is a luer taper. In the peritoneal dialysis device for animals 1 shown in FIG. 1, the portion of the waste fluid container connector 31 connected to the waste fluid catheter 30 is a male connector, and the portion of the waste fluid container connector 31 connected to the waste fluid container 33 is a female connector. Alternatively, the portion of the waste fluid container connector 31 connected to the waste fluid catheter 30 may also be a female connector, and the portion of the waste fluid container connector 31 connected to the waste fluid container 33 may also be a male connector.

In one or more embodiments, the waste fluid catheter 30 further comprises a waste fluid catheter clamp 32 disposed on the waste fluid catheter 30 and opens or closes the passageway of the waste fluid catheter 30. However, it may not be necessary to use the catheter clamp 32 to open or close the passageway of the waste fluid catheter 30. The catheter may also be manually squeezed by a machine or a care worker to close the passageway of the waste fluid catheter 30, or controlled by a valve disposed inside the waste fluid catheter 30 to open or close the passageway of the catheter 30 (not shown). In one or more embodiments, the waste fluid catheter 30 includes a sampling port 34, which allows a doctor or a care worker to sample the waste fluid. When the sampling port 34 is provided in the injection catheter 20, the sample is diluted by the injected dialysate, because the injection catheter 20 is used for both injecting the dialysate and drawing out the waste fluid. When the sampling port 34 is provided in the waste fluid catheter 30, the sample will not be diluted by the dialysate and the sampling will be more accurate, because the waste fluid catheter 30 is only used for transporting the waste fluid.

The syringe catheter 40 is used for transporting the dialysate in the four-way joint 50 to the syringe 41, and transporting the dialysate flowing out from the syringe 41 to the four-way joint 50. The barrel of the syringe 41 is marked with a scale for quantifying the amount of dialysate injected.

In the peritoneal dialysis device for animals 1 of one or more embodiments disclosed herein, the dialysate is drawn up into the syringe 41 before injected into the peritoneum, and the amount of the dialysate is precisely quantified on the scale of the syringe 41, to overcome the low accuracy of the conventional peritoneal dialysis device. Then the care worker or machine slowly but stably injects the dialysate in the syringe 41 through the catheters and connectors into the peritoneal cavity by applying a steady force to the plunger of the syringe 41, to overcome the instability that exists within peritoneal dialysis devices that rely on gravity to push the fluid in the catheter. When the syringe 41 is installed on a machine which can apply a stable force to push the plunger of the syringe slowly and at a constant speed, the peritoneal dialysis device for animals can automatically inject the dialysate without the need for human care workers to perform and supervise personally, thus saving a lot of manpower.

One or more embodiments of the method for using the peritoneal dialysis device for animals is disclosed below.

Step S100: The dialysate is drawn up and the amount of the dialysate to be injected is quantified. The machine or care worker closes the passageway of the injection catheter 20 and the waste fluid catheter 30 by means of the catheter clamps 22, 32, valves, or directly squeezing the catheter, and pulls the plunger of the syringe 41 to create negative pressure to draw up the dialysate out of the dialysate container. The dialysate flowing out from the dialysate container sequentially passes through the dialysate container connector 11, the dialysate catheter 10, the four-way joint 50 and the syringe catheter 41 before entering into the syringe 41.

Step S200: The dialysate is injected into the peritoneal cavity of the patient. The machine or care worker closes the passageway of the dialysate catheter 10 and the waste fluid catheter 30 by means of the catheter clamps 12, 32, valves or directly squeezing the catheter, and opens the passageway of the injection catheter 20 by means of the catheter clamp 22, a valve or directly releasing the catheter and pushes the plunger of the syringe 41 to force the dialysate in the syringe 41 to flow out. The dialysate flowing out from the syringe 41 sequentially passes through the syringe catheter 40, the four-way joint 50, the injection catheter 20, the injection catheter connector 21, and the peritoneal catheter 24 before entering into the peritoneal cavity of the patient, allowing the dialysate to contact the patients peritoneum for a period of time. Using the peritoneum as a dialysis semi-permeable membrane, the body wastes are removed and transported to the dialysate of the peritoneal cavity.

Step S300: The waste fluid containing body wastes from the patient's peritoneal cavity is drained to the waste fluid container 33. The machine or care worker opens the passageway of the waste fluid catheter 30 by means of the catheter clamp 32, a valve, or directly releasing the catheter. As the dialysate catheter 10 is already closed, and the plunger of the syringe 41 has been pushed down to the bottom, so the pressure in the peritoneal cavity pushes the waste fluid containing body wastes toward the direction of the waste fluid container 33. However, whether the waste fluid is discharged and the amount of the waste fluid discharged may also be controlled by pulling/pushing of the syringe by the care worker. The waste fluid flowing out from the peritoneal cavity sequentially passes through the peritoneal catheter 24, the injection catheter connector 21, the injection catheter 20, the four-way joint 50, the waste fluid catheter 30, and the waste fluid container connector 31 before entering into the waste fluid container 33.

According to the structure and method described above, one or more embodiments disclosed herein provide a peritoneal dialysis device for animals which can accurately quantify the amount of the dialysate injected, slowly but continuously and stably inject the dialysate, automatically inject the dialysate, and accurately quantify the amount of the waste fluid discharged, without the need for human care workers to perform and supervise personally, thus saving a lot of manpower.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A peritoneal dialysis device for animals, comprising:
a dialysate catheter, that is connected at a first end to a dialysate container connector and at a second end to a four-way joint;
an injection catheter, that is connected at a first end to an injection catheter connector and at a second end to the four-way joint;
a waste fluid catheter, that is connected at a first end to a waste fluid container connector and at a second end to the four-way joint; and
a syringe catheter, that is connected at a first end to a syringe and at a second end to the four-way joint.

2. The peritoneal dialysis device for animals according to claim 1, wherein the dialysate catheter is connected to a dialysate container through the dialysate container connector;
the injection catheter is connected to a peritoneal catheter through the injection catheter connector; and
the waste fluid catheter is connected to a waste fluid container through the waste fluid container connector.

3. The peritoneal dialysis device for animals according to claim 1, wherein each of the dialysate container connector, the injection catheter connector and the waste fluid container connector is a luer taper.

4. The peritoneal dialysis device for animals according to claim 1, wherein the injection catheter comprises a waterwheel that rotates in response to a flow of fluid in the injection catheter.

5. The peritoneal dialysis device for animals according to claim 1, wherein the waste fluid catheter comprises a sampling port.

6. The peritoneal dialysis device for animals according to claim 1, wherein the dialysate catheter comprises a dialysate catheter clamp disposed on the dialysate catheter, and the dialysate catheter clamp opens or closes a passageway of the dialysate catheter;
the injection catheter comprises an injection catheter clamp disposed on the injection catheter, and the injection catheter clamp opens or closes a passageway of the injection catheter; and
the waste fluid catheter comprises a waste fluid catheter clamp disposed on the waste fluid catheter, and the waste fluid catheter clamp opens or closes a passageway of the waste fluid catheter.

7. The peritoneal dialysis device for animals according to claim 1, wherein
the dialysate catheter comprises a multi-way joint including at least three ends, wherein at least two of the at least three ends are connected to a plurality of the dialysate container connectors, and a remaining of the at least three ends is connected to the four-way joint.

8. The peritoneal dialysis device for animals according to claim 7, wherein
the dialysate catheter is connected to a plurality of dialysate containers through the plurality of the dialysate container connectors,

9. The peritoneal dialysis device for animals according to claim 2, wherein
the injection catheter connector is connected to the peritoneal catheter through an isolating short tube, so as to prevent the injection catheter connector from being directly connected to the peritoneal catheter.
